(19) United States
(12) Patent Application Publication
(10) Pub. No.: US 2021/0001133 A1
(43) Pub. Date: Jan. 7, 2021

(54) System for Managing Clinical Data

(71) Applicant: Saluda Medical Pty Ltd, Artarmon (AU)

(72) Inventors: Matthew Marlon Williams, Artarmon (AU); Dean Michael Karantonis, Artarmon (AU); Ivan Guelton, Artarmon (AU); Robert Bruce Gorman, Artarmon (AU)

(73) Assignee: Saluda Medical Pty Ltd, Artarmon (AU)

(21) Appl. No.: 17/040,521

(22) PCT Filed: Mar. 23, 2018
(86) PCT No.: PCT/AU2018/050278

§ 371 Date: Sep. 22, 2020

(51) Int. Cl. A61N 1/372; G16H 40/67; G16H 20/70; A61N 1/36

(52) U.S. Cl. CPC A61N 1/37282; G16H 40/67; A61N 1/37247; A61N 1/36135; G16H 20/70

(57) ABSTRACT

A system for managing clinical data of an implanted neuromodulation device. An implanted neuromodulation device delivers electrical neurostimulation therapy, and captures recordings of neural responses to the electrical neurostimulation therapy. The device communicates clinical data including recordings of the neural responses to a non-implanted receiver. A supervisory device receives clinical data from the implanted neuromodulation device via the receiver, and presents the clinical data for review by a reviewer.

### TECHNICAL FIELD

The present invention relates to neurostimulation, and in particular to a management system and method for retrieving clinical data from an implanted neurostimulation device and for optimising and efficiently presenting such clinical data.

### BACKGROUND OF THE INVENTION

There are a range of situations in which it is desirable to apply neural stimuli in order to give rise to a compound action potential (CAP). For example, neuromodulation is used to treat a variety of disorders including chronic pain, Parkinson's disease, and migraine. A neuromodulation system applies an electrical pulse to tissue in order to generate a therapeutic effect. When used to relieve chronic pain, the electrical pulse is applied to the dorsal column (DC) of the spinal cord, referred to as spinal cord stimulation (SCS). Neuromodulation systems typically comprise an implanted electrical pulse generator, and a power source such as a battery that may be rechargeable by transcutaneous inductive transfer. An electrode array is connected to the pulse generator, and is positioned in the dorsal epidural space above the dorsal column. An electrical pulse applied to the dorsal column by an electrode causes the depolarisation of neurons, and generation of propagating action potentials. The fibres being stimulated in this way inhibit the transmission of pain from that segment in the spinal cord to the brain. To sustain the pain relief effects, stimuli are applied substantially continuously, for example at a frequency in the range of 50-100 Hz.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood that the element may be any one of the listed options, or may be any combination of two or more of the listed options.

### SUMMARY OF THE INVENTION

According to a first aspect the present invention provides a system for managing clinical data of an implanted neuromodulation device, the system comprising:

an implanted neuromodulation device configured to deliver electrical neurostimulation therapy, and configured to capture recordings of neural responses to the electrical neurostimulation therapy, and configured to communicate clinical data including recordings of neural responses to a non-implanted receiver; and

a supervisory device configured to receive clinical data from the implanted neuromodulation device via the receiver, and to present the clinical data for review by a reviewer.

According to a second aspect the present invention provides a method of managing clinical data of an implanted neuromodulation device, the method comprising:

an implanted neuromodulation device delivering electrical neurostimulation therapy;

the implanted neuromodulation device capturing recordings of neural responses to the electrical neurostimulation therapy;

communicating clinical data including the recordings of neural responses to a non-implanted receiver; and

receiving the clinical data from the implanted neuromodulation device via the receiver, and presenting the clinical data for review by a reviewer.

According to a third aspect the present invention provides a non-transitory computer readable medium for managing clinical data of an implanted neuromodulation device, comprising instructions which, when executed by one or more processors, causes performance of the following:

receiving clinical data from the implanted neuromodulation device, the clinical data including recordings captured by the implanted neuromodulation device of neural responses to electrical neurostimulation therapy delivered by the implanted neuromodulation device; and

presenting the clinical data for review by a reviewer.

In some embodiments of the invention, the implanted neuromodulation device is configured to record the clinical data substantially continuously during device operation. For example, in some embodiments of the invention the implanted neuromodulation device is configured to record the clinical data for a period of at least 8 hours of device operation. In some embodiments of the invention the implanted neuromodulation device is configured to record the clinical data for a period of at least 2 days of device operation. In some embodiments of the invention the implanted neuromodulation device is configured to record the clinical data for a period of at least 5 days of device operation.

In some embodiments of the invention the implanted neuromodulation device is configured to communicate all recorded clinical data to the non-implanted receiver whenever within wireless communications range of the non-implanted receiver. In some embodiments of the invention the implanted neuromodulation device is configured to delete all recorded clinical data once the recorded data is communicated to the receiver.

In some embodiments of the invention the implanted neuromodulation device is further configured to record clinical data comprising user commands input to the implanted neuromodulation device. In some embodiments of the invention the implanted neuromodulation device is further configured to record clinical data comprising stimulus current, feedback target, battery state and the like.

In some embodiments of the invention the supervisory device is configured to present the clinical data for review by presenting a subset of the clinical data as selected by filtering by time range. In some embodiments of the invention the filtering is effected by selecting from a predefined list of different time ranges. In some embodiments of the invention the filtering is effected by the reviewer defining a custom time period. In some embodiments of the invention the filtering is effected by a graphical user interface presenting a slider bar permitting the reviewer to define start and/or end times for review.

In some embodiments of the invention the supervisory device is configured to present statistics derived from the clinical data. In some embodiments of the invention the statistics are recalculated for a chosen time period, at times when the reviewer selects a subset of the clinical data for display.

In some embodiments of the invention the clinical data comprises logged events, which are presented for review by an indicia, and wherein each such indicia may be clicked or tapped to reveal a summary or full detail of the respective logged event represented by that indicia. In some embodiments of the invention the logged events comprise one, or more than one, or all, of a Patient Command event category, an Error event category, an Exception event category, a Stimulator Response event category, a Programming Command event category, a Battery event category and an Other event category.

In some embodiments of the invention therapy logs are displayed. In some embodiments of the invention a therapy log comprises a plurality of histograms of a therapy parameter. In some embodiments of the invention the therapy parameter comprises feedback variable measurements obtained by the implant, each histogram representing a subset of the clinical data. In some embodiments of the invention the histograms are represented on a timescale by use of a heat map representation of the histograms. In some embodiments of the invention the feedback variable is an ECAP magnitude. In some embodiments of the invention the therapy parameter comprises a feedback target. In some embodiments of the invention the therapy parameter comprises a stimulus current value. In some embodiments of the invention the presented clinical data includes device programming data. In some embodiments of the invention the presented clinical data includes device programming data in the form of programming notes entered by a clinician during device programming.

In some embodiments of the invention the presented clinical data includes an ECAP recording trace. In some embodiments of the invention the presented clinical data includes a plurality of ECAP recording traces.

In some embodiments of the invention the presented clinical data includes quantitative analysis of one or more histograms of a therapy parameter.

### BRIEF DESCRIPTION OF THE DRAWINGS

An example of the invention will now be described with reference to the accompanying drawings, in which:

FIG. 1 schematically illustrates an implanted spinal cord stimulator;

FIG. 2 is a block diagram of the implanted neurostimulator;

FIG. 3 is a schematic illustrating interaction of the implanted stimulator with a nerve;

FIG. 4 illustrates the architecture of a system for managing clinical data of an implanted neuromodulation device in accordance with an embodiment of the invention;

FIG. 5 further illustrates the architecture of the system of FIG. 4;

FIG. 6 illustrates the presentation of clinical data gathered over a first time period, effected by the clinical programming application in accordance with an embodiment of the invention;

FIG. 7 illustrates the presentation of clinical data gathered over a five minute time period, effected by the clinical data viewer in accordance with an embodiment of the invention;

FIG. 8 illustrates the presentation of clinical data gathered over an eight week time period, effected by the clinical data viewer in accordance with an embodiment of the invention;

FIGS. 9a and 9b illustrate the clinical data viewer page menu;

FIG. 10 is a partial view of the clinical data viewer;

FIG. 11a and FIG. 11b illustrate the presentation and calculation of battery statistics by the clinical data viewer in accordance with an embodiment of the invention

FIG. 12 illustrates the Histograms tab of the clinical data viewer;

FIG. 13a illustrates the Histogram Details page, and FIG. 13b shows the architecture of view controllers and their related models and views;

FIG. 14 illustrates the Programming Session Page;

FIG. 15 illustrates the system components for displaying ECAPs;

FIG. 16 displays how to calculate the number of stimulations where the FBV falls within a certain range;

FIG. 17 illustrates ECAP viewing within the clinical data viewer;

FIG. 18 illustrates the Posture Change Assessment page of the clinical data viewer

FIG. 19 illustrates the Patient Dashboard Interface page of the clinical data viewer

FIG. 20 illustrates pre-processing raw usage log data before display by the clinical data viewer;

FIG. 21 illustrates timestamp correction; and

FIGS. 22-25 illustrate histogram analysis for automated output of quantitative responses.

### DESCRIPTION OF THE PREFERRED EMBODIMENTS

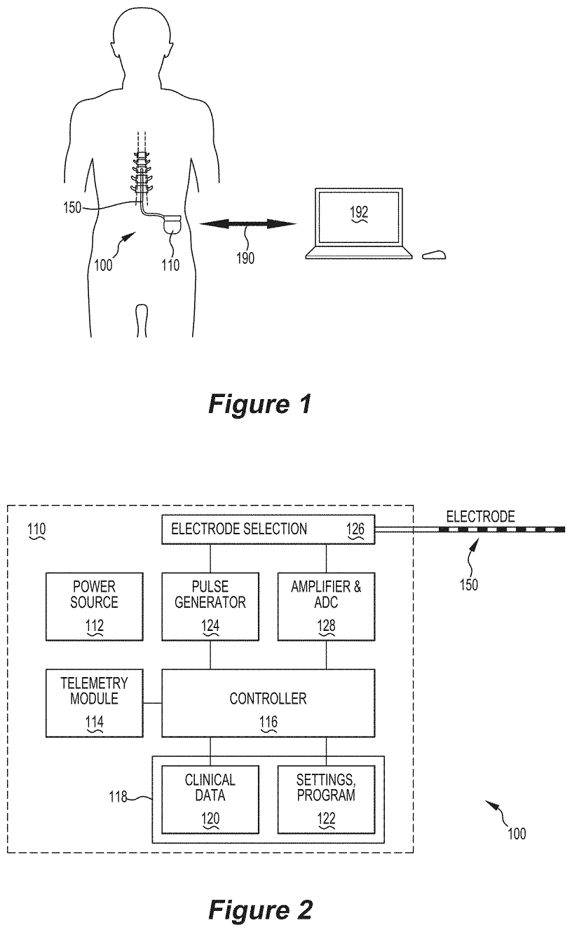

FIG. 1 schematically illustrates an implanted spinal cord stimulator 100. Stimulator 100 comprises an electronics module 110 implanted at a suitable location in the patient's lower abdominal area or posterior superior gluteal region, and an electrode assembly 150 implanted within the epidural space and connected to the module 110 by a suitable lead. Numerous aspects of operation of implanted neural device 100 are reconfigurable by an external control device 192. Moreover, implanted neural device 100 serves a data gathering role, with gathered data being communicated to external device 192 via any suitable transcutaneous communications channel 190.

FIG. 2 is a block diagram of the implanted neurostimulator 100. Module 110 contains a battery 112 and a telemetry module 114. In embodiments of the present invention, any suitable type of transcutaneous communication 190, such as infrared (IR), electromagnetic, capacitive and inductive transfer, may be used by telemetry module 114 to transfer power and/or data between an external device 192 and the electronics module 110. Module controller 116 has an associated memory 118 storing clinical data 120 as is the focus of the present invention, but also storing patient settings and control programs 122, and the like. Controller 116 controls a pulse generator 124 to generate stimuli in the form of current pulses in accordance with the patient settings and control programs 122. Electrode selection module 126 switches the generated pulses to the appropriate electrode(s) of electrode array 150, for delivery of the current pulse to the tissue surrounding the selected electrode(s). Measurement circuitry 128 is configured to capture measurements of neural responses sensed at sense electrode(s) of the electrode array as selected by electrode selection module 126, and the controller 116 stores such neural response measurements in the clinical data storage 120, whether in the form of a time-limited voltage trace, a single measure of ECAP amplitude, or in any other form which captures the desired information pertaining to the evoked ECAP or absence thereof

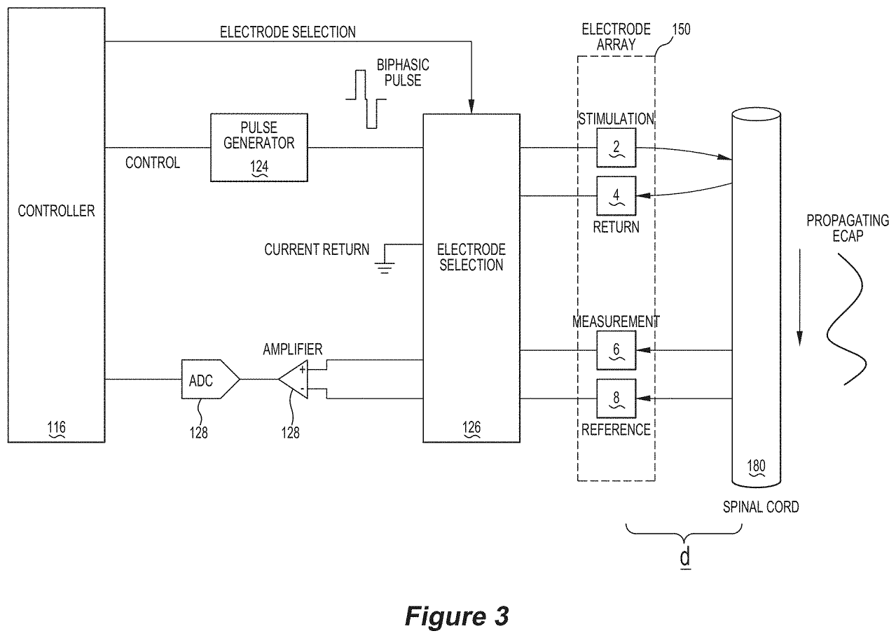

FIG. 3 is a schematic illustrating interaction of the implanted stimulator 100 with a nerve 180, in this case the spinal cord however alternative embodiments may be positioned adjacent any desired neural tissue including a peripheral nerve, visceral nerve, parasympathetic nerve or a brain structure. Electrode selection module 126 selects a stimulation electrode 2 of electrode array 150 to deliver an electrical current pulse to surrounding tissue including nerve 180, and also selects a return electrode 4 of the array 150 for stimulus current recovery to maintain a zero net charge transfer.

Delivery of an appropriate stimulus to the nerve 180 evokes a neural response comprising a compound action potential which will propagate along the nerve 180 as illustrated, for therapeutic purposes which in the case of a spinal cord stimulator for chronic pain might be to create paraesthesia at a desired location. To this end the stimulus electrodes are used to deliver stimuli at any therapeutically suitable frequency, for example 30 Hz, although other frequencies may be used including as high as the kHz range, and/or stimuli may be delivered in a non-periodic manner such as in bursts, or sporadically, as appropriate for the patient. To fit the device, a clinician applies stimuli of various configurations which seek to produce a sensation that is experienced by the user as a paraesthesia. When a stimulus configuration is found which evokes paraesthesia, which is in a location and of a size which is congruent with the area of the user's body affected by pain, the clinician nominates that configuration for ongoing use.

The device 100 is further configured to sense the existence and intensity of compound action potentials (CAPs) propagating along nerve 180, whether such CAPs are evoked by the stimulus from electrodes 2 and 4, or otherwise evoked. To this end, any electrodes of the array 150 may be selected by the electrode selection module 126 to serve as measurement electrode 6 and measurement reference electrode 8. Signals sensed by the measurement electrodes 6 and 8 are passed to measurement circuitry 128, which for example may operate in accordance with the teachings of International Patent Application Publication No. WO2012155183 by the present applicant, the content of which is incorporated herein by reference. The output of circuitry 128 is used by controller 116 in a feedback arrangement to control the application of subsequent stimuli, and the controller 116 also stores the recording of the neural response or one or more parameters thereof such as ECAP amplitude to the Clinical Data storage 120. There are a range of circumstances in which it is desirable to obtain an electrical measurement of a compound action potential (CAP) evoked on a neural pathway by an electrical stimulus applied to the neural pathway. As the neural response can be contemporaneous with the stimulus and/or the stimulus artefact, CAP measurements present a difficult challenge of implant design. However, the present invention recognises that as this problem is solved, large amounts of clinical data comprising ECAP measurements and other device parameters can be generated by an implanted neurostimulation device and stored in the Clinical Data storage 120. Accordingly, the present invention provides for clinical data to be captured by the device 100 while in the field, and also while in a clinical setting, in a manner which is battery efficient and thus conducive to field deployment. By providing a system for capture of such field data the present invention provides for substantially improved monitoring of device operation, therapeutic efficacy, compliance with operating limits, and the like. Such data can be captured and stored while the implant recipient goes about normal daily activities for later retrieval and analysis, rather than only capturing such data in a time-limited manner in the artificial setting of a clinical attendance.

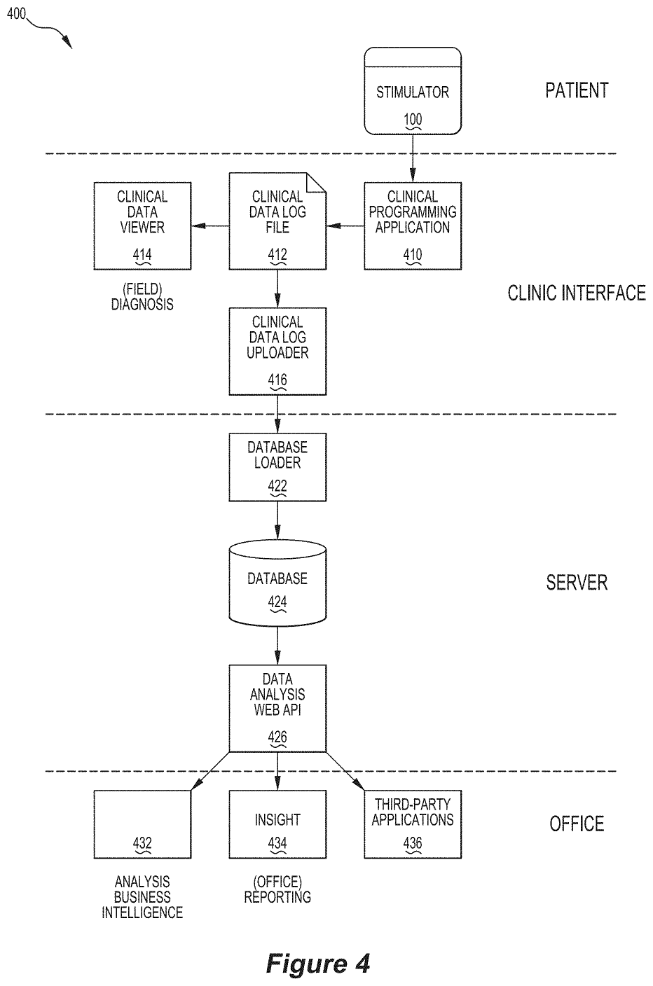

FIG. 4 illustrates the architecture of a system 400 for managing clinical data of implanted neuromodulation device 100 in accordance with a first embodiment of the invention. Stimulator 100 applies stimuli over a potentially long period such as weeks or months and records neural responses, stimulation settings, paraesthesia target level, and other operational parameters, discussed further below. The stimulator 100 comprises a Closed Loop Stimulator (CLS), in that the recorded neural responses are used in a feedback arrangement to control stimulation settings on a continuous or ongoing basis. To effect suitable SCS therapy stimulator 100 may deliver tens, hundreds or even thousands of stimuli per second, for many hours each day. The feedback loop may operate for most or all of this time, by obtaining neural response recordings following every stimulus, or at least obtaining such recordings regularly. Each recording generates a feedback variable such as a measure of the amplitude of the evoked neural response, which in turn results in the feedback loop changing the stimulation parameters for a following stimulus. Stimulator 100 thus produces such data at a rate of tens or hundreds of Hz, or even kHz, and over the course of hours or days this process results in large amounts of clinical data. This is unlike past neuromodulation devices such as SCS devices which lack any ability to record any neural response.

When brought in range with a receiver, stimulator 100 transmits data via telemetry module 114 to a clinical programming application 410 of a clinic interface, which compiles a clinical data log file 412 which is manipulated and optimised and efficiently presented by a clinical data viewer 414, for field diagnosis by a clinician, field clinical engineer (FCE) or the like.

The clinical data viewer 414 is a software application used to analyse data generated by the stimulator 100. The clinical data viewer is installed on a Clinical Interface (CI) tablet computer. The clinical data viewer may be installed by trained staff on the CI tablet, or on another computer running any suitable operating system such as Microsoft Windows. Data is collected from the stimulator 100 by the Clinical Programming Application (CPA) 410. The data can be grouped into two main sources: 1. Data collected in real-time during a programming session, and 2. Data downloaded from a stimulator after a period of non-clinical use by a patient.

This data is stored by the CPA 410 in a Clinical Data Log (.h5) file 412. The Clinical Data Viewer 414 can open one Clinical Data Log file at a time. The flow of data is shown in FIG. 4. The Clinical Data Uploader 416 is an application that runs in the background on the CI, that automatically uploads files generated by the CPA 410 to a file server. The Data Log Loader 422 is a service which runs on the data server, and monitors the patient data folder for new files and when Clinical Data Log files are uploaded by the Clinical Data Uploader 416 it extracts the data and pushes it to a Database 424.

All logs downloaded from the stimulator 100 are compressed by use of a suitable data compression technique before transmission by telemetry module 114, to enable faster download speeds for logs and/or before storage into the Clinical Data storage 120 so as to allow storage by stimulator 100 of higher resolution data, to provide more data for post-analysis and more detailed data mining for events during use. The server 422, 424, 426 may be configured to provide the user or clinician with a summary of device usage, therapy output, and errors, in a simple single-view page immediately after logs are downloaded upon device connection. The ability to obtain, store, download and analyse such large amounts of neuromodulation data means that the present embodiment can improve patient outcomes in difficult conditions, enable faster, more cost effective and more accurate troubleshooting and patient status, and enable the gathering of statistics across patient populations for later analysis, with a view to diagnosing aetiologies and predicting patient outcomes.

A key component of the system 400 is the Clinical Data Viewer 414, a software application that facilitates the analysis of data stored in Clinical Data Log files 412. The Clinical Data Viewer 414 is intended to be used in the field to diagnose patient issues and optimise therapy for the patient.

A number of abbreviations and terms require definition. A Column refers to a column of data in a table. Data for a particular variable are stored in, and referred to as, a column within a table. To select a particular type of data to view in the CDV 414 is to select that data type's column. An Error Log is a time-based record of error events which can be downloaded from the stimulator 100 during a programming session with a clinician or field clinical engineer (FCE). The Reference Current is the current produced by a single current source output of stimulator 100. The Reference Current cannot exceed 12.5 mA (the maximum of a single current source output). Unless otherwise stated, the values returned in the data logs 412 are in terms of reference current. Stimulation refers to a pulse which is typically bi-phasic or tri-phasic. A stimulation refers to the delivery of one of these pulses. Total Current is the current level displayed in the CPA 410 and noted down by the FCE on programming worksheets. If enough information is available regarding the stimulation setup, the Total Current can be calculated using the Reference Current. For example, when there are 2 stimulus electrodes set to 50% each (on the CPA) and the Reference Current is 5 mA, the Total Current would be 10 mA. A Therapy Log is a compressed record of each stimulation which can be downloaded from the storage 120 of stimulator 100 during a programming session. A Usage Log is a time-based record of events which can be downloaded from the storage 120 of stimulator 100 during a programming session. The Usage Log records patient and clinician interactions with the stimulator 100, as well as the response from the stimulator 100 to such interactions. A Feedback Variable (FBV) is the value that represents a measure of the amplitude of the ECAP signal. Clinical Data Log 412 contains numerous Data Logs, including Data Logs for each type of data downloaded from the stimulator (e.g. Usage Logs, Histograms, etc.).

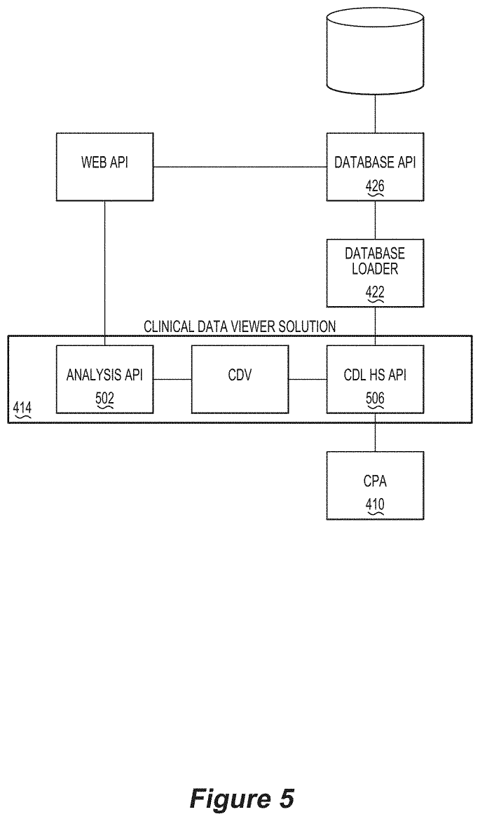

The Clinical Data Viewer 414 is written to allow easy integration into the Clinical Programming Application. FIG. 5 shows the Data Analysis Suite of software modules, and highlights the modules that are used by the Clinical Data Viewer 414. Independent software modules are used to maximise code reuse and simplify testing across the software tools.

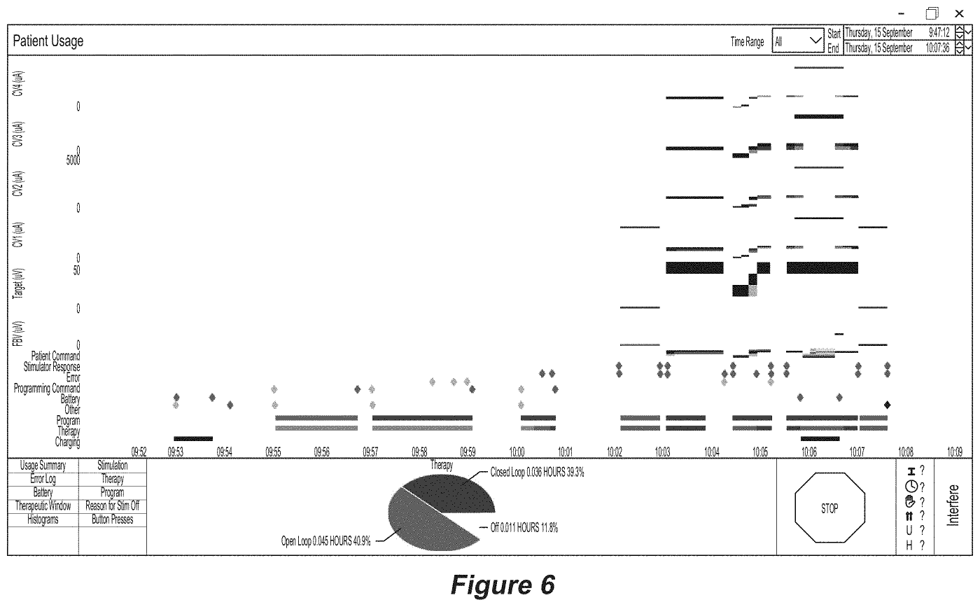

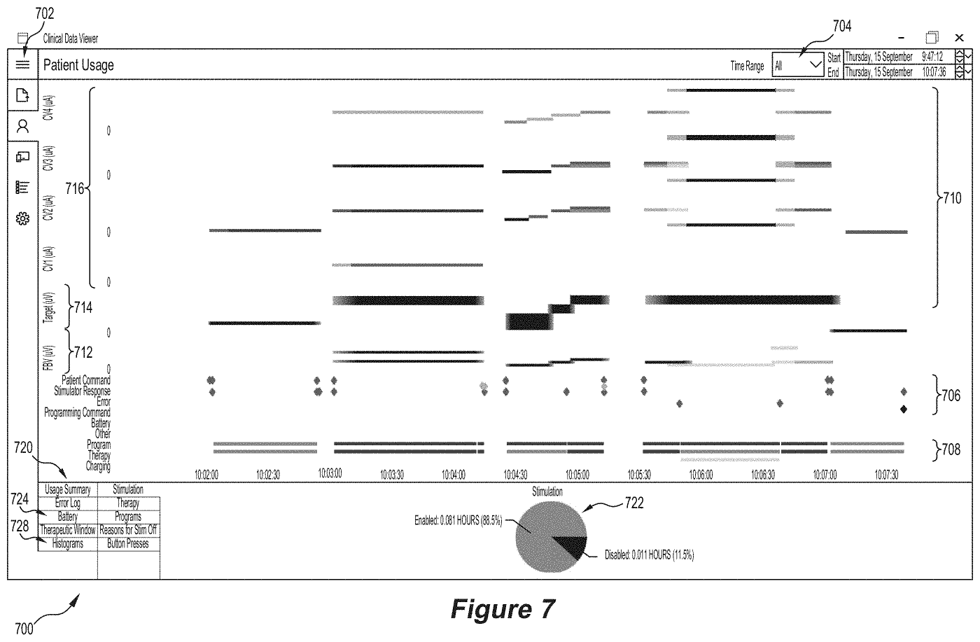

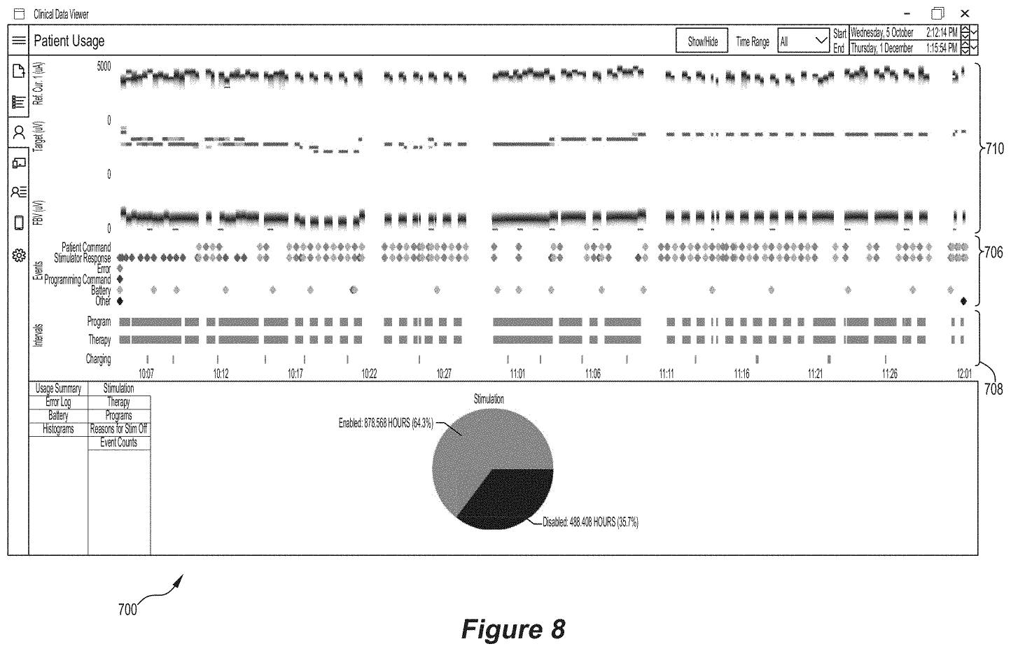

FIG. 6 illustrates the presentation of the clinical data from storage 120, for a clinical usage period of about 17 minutes, effected by clinical programming application 410 in accordance with the present embodiment of the invention. FIG. 7 illustrates the presentation of the clinical data from storage 120, for a non-clinical usage period of about five minutes, effected by the Clinical Data Viewer 414 in accordance with the present embodiment of the invention. FIG. 8 illustrates the presentation of the clinical data from storage 120, for a non-clinical usage period of about eight weeks, effected by the Clinical Data Viewer 414 in accordance with the present embodiment of the invention. The Clinical Data Viewer 414 can have one Clinical Data Log file 412 open at a time, however multiple instances of the application 414 may be run at once. Patient Usage Data (i.e. Usage Logs, Error Logs and Therapy Logs) is loaded by the PatientUsageDataProvider class which loads the data, filters out corrupted data, fixes timestamps and generates processed data such as the BatteryChargeIntervalCollection and StimulationlntervalCollection using functions in the Data Analysis API 502. To do this, the raw data in the H5 files must be converted into a standard format. This is done by classes in the ClinicalDataLogH5Parser module 506.

The Clinical Data Viewer application 414 follows the Model-View-View-Model design pattern, allowing the business logic (i.e. the Data Analysis API 502, FIG. 5) to be separated from the logic that describes how the data is displayed. The Clinical Data Viewer 414 uses WPF Data Binding to relay notifications between the views and their view-models.

The MainWindow 700 shown in FIGS. 7 and 8 is the primary view for the application 414 and is bound to the ClinicalDataViewer. The main window 700 contains a <Frame> control which displays one of possible pages provided by the PageController. Each page is derived from the CustomPage class, which allows uniformity across the common page features such as the title page, back button and custom commands. The main window 700 controls the navigation between pages. Each of these pages is bound to its own view controller, each of which are a property of the parent view controller, the ClinicalDataViewer, except for the DataLogTableViewModel which is created for a specific DataLog.

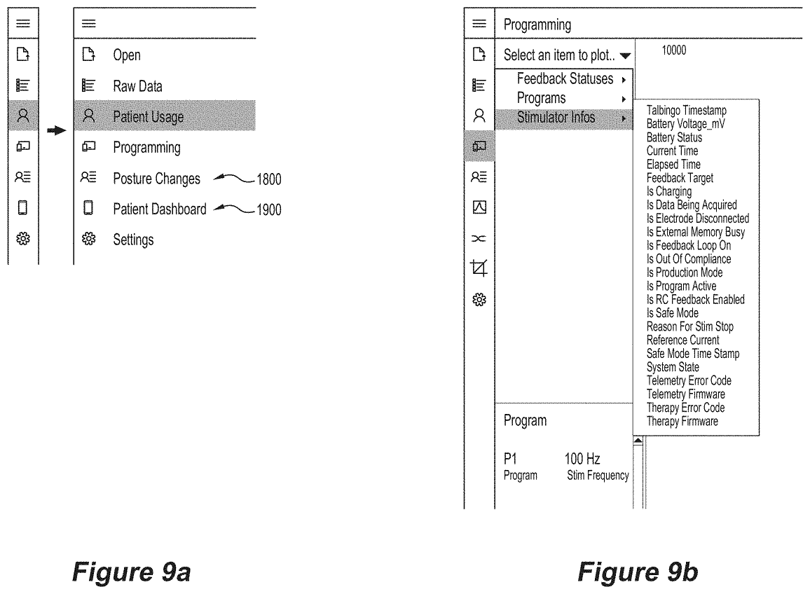

The Clinical Data Viewer 414 has an expandable menu 702 which controls navigation between different pages. The menu 702 can be expanded by pressing the ≡icon, as shown in FIGS. 9a and 9b. FIG. 9b in particular shows the means by which the reviewer may select the types of data to be displayed or plotted in the clinical data viewer 414.

The Patient Usage Page of FIGS. 7 & 8 displays data from Usage Logs, Therapy Logs and Error Logs downloaded from stimulator 100 during a programming session with the CPA 410, and is used to analyse how the patient has been using the stimulator 100 outside the clinic. The Patient Usage Page is particularly advantageous in assessing how the patient has been using the stimulator 100 in the field, as explained in further detail in the following.

Patient Usage data can be filtered by a custom or pre-defined time range by selecting a time range from the drop-down menu 704. Selecting a pre-defined time range (One Day, One Week, One Month or Three Months) will select all the data in the range ending at the most recent data point. If a Custom time range is selected, the start and end points can be chosen by the user. If the user decides to zoom in on a particular area of the data, they can set the time range to the area in view. When a time range is selected, all data not in that time range is hidden from display and all statistics are re-calculated for the selected time range.

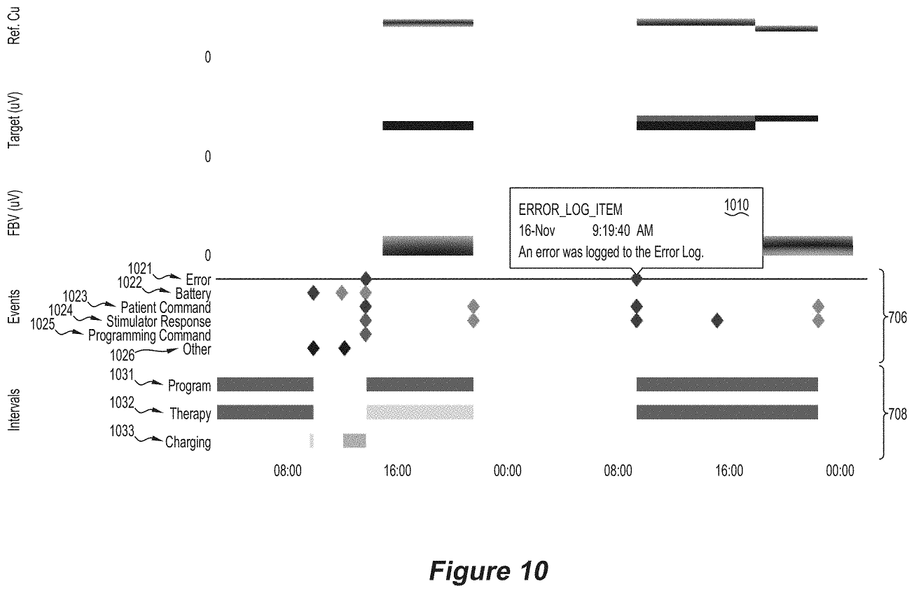

Each logged event (i.e. a Usage Log item or Error Log item) is displayed on the Patient Usage Page in the field 706 as a diamond and is plotted against time (x-axis). Clicking on a diamond opens a pop-up 1010 which displays the raw data associated with that event, as shown in FIG. 10. Events are grouped into seven categories (one per row), of which only 6 are populated in the data set shown and thus only 6 categories are shown in field 706 in FIGS. 6-10. Each category has a set colour per event type.

In more detail, the categories of logged events are as follows. The Patient Command event category 1023 comprises Commands sent to the stimulator from the Remote Control, including: Select Program; Therapy Stop; Therapy Off; Increased Amplitude and Decrease Amplitude, each of which are represented by a respective colour in the category line 1023 as shown in FIG. 10. The Error event category 1021 comprises error log items including out of compliance, electrode disconnection, and error log item. The Exception event category comprises exception log items including Telemetry Down Mode, Therapy Down Mode, and Exception Log Item. No Exceptions occurred in the data set of FIGS. 7-10 and so no Exception line is shown.

The Stimulator Response event category 1024 comprises Acknowledgements from the stimulator that a Patient Command or Programming Command has been acted upon, including: Amplitude Feedback ON Change; Amplitude Feedback OFF change; Therapy Finish; Therapy Start Feedback On, Therapy Start Feedback Off, and Feedback Loop. The Programming Command event category 1025 comprises Commands sent to the stimulator from the Clinical Programming Application, including: Therapy Stop Order and Device Programmed. The Battery event category 1022 comprises Battery charging events, including: Disconnect Battery; High Temperature; Charge Start; Battery Already Charged; Successful Charge; Charge Timeout and Charge Finish. The Other event category 1026 comprises Other events logged by the stimulator that do not affect the therapy, including: Set Time; Erase; Reset; Safe Mode Recharge Enabled; Hardware Reset; Clear; Extra Data; Sequence Number; New RTC ID; Filler, and No Event.

In FIGS. 7-10, Intervals 708 are also calculated and shown. Intervals 708 represent the time between events which correspond to a single system state. The Clinical Data Viewer 414 calculates the system state for each interval 708 based on the events preceding it. Intervals 708 are displayed on the Patient Usage Page as coloured bars plotted against time (x-axis), as shown in FIGS. 7-10. Clicking on the bar representing any given Interval opens a pop-up which displays detailed information about the state of the stimulator at that time. Intervals 708 are grouped into three categories 1031, 1032, 1033, as shown in FIG. 10. The Program Interval 1031 indicates the current program that the patient was using. Within this category, four colours are used to represent which of four programs were chosen, and with no colour indicating stimulation off. The Therapy interval 1032 indicates the state of the Therapy, with colours used to indicate Open loop stimulation, closed loop stimulation or stimulation off. The Charging Interval 1033 indicates when the stimulator was charging.

In FIGS. 7-10, Therapy Logs 710 are displayed, in the form of a heat-map. Each heat map is a representation of the proportion of stimuli (colour intensity) which had a particular magnitude (y-axis) at each time point (x-axis). The more intense the colour, the greater the proportion of stimuli with that magnitude at that time. There will be at least three heat maps displayed (Feedback Variable 712, Feedback Target 714, and Current 716) with additional current heat maps for each additional stim set in use.

In FIG. 7, the data displayed in the Usage Summary tab 720 is obtained from the stimulation intervals, as follows:

| | |
|---|---|
| Stimu- | The total duration and percentage of time spent with |
| lation | stimulation on (enabled) and off (disabled). |
| Therapy | The total duration and percentage of time spent in each |
| | therapy mode (i.e. closed loop, open loop, off). |
| Programs | The total duration and percentage of time spent in each |
| | program. |
| Reasons | Every time stimulation is stopped a LOG_THERAPY_FINISH |
| for Stim | is logged. Each of these events has a reason for stimulation |
| Off | stopping attributed to it. This section groups the |
| | LOG_THERAPY_FINISH events by reason for stimulation |
| | stopping and shows the total number of events for each group. |
| Event | The number of times each of the following events occurred: |
| Counts | Amplitude changed when in Closed Loop |
| | Amplitude changed when in Open Loop |
| | Program changed |
| | Stop Button pressed |
| | ‘+’ Button pressed |
| | ‘−’ Button pressed |

The Usage Summary plots are displayed as a pie-chart 722 by default, however there are other plotting and data options available by right-clicking on the plot.

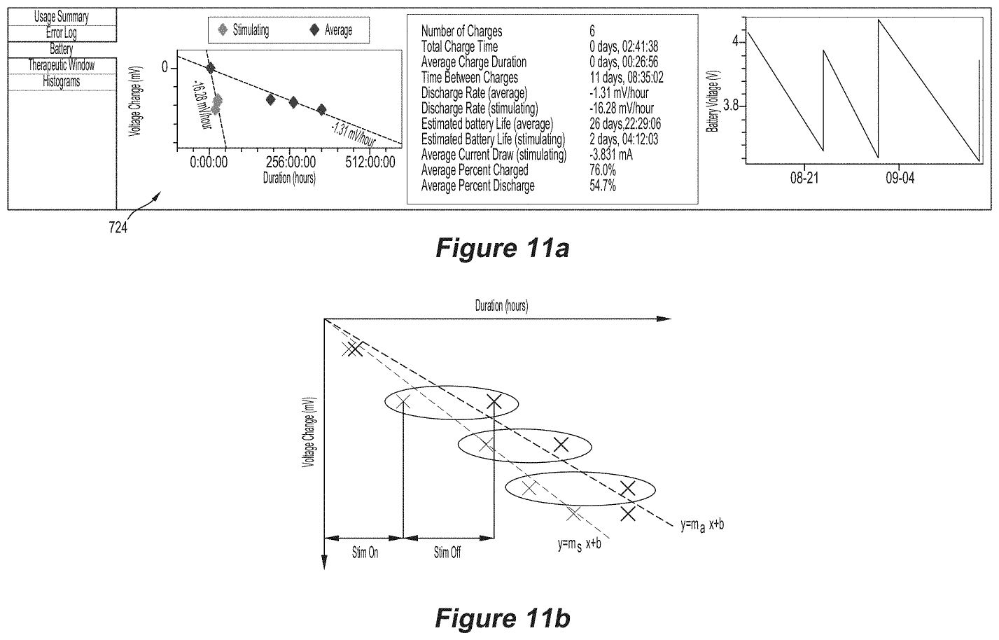

The Battery tab 724 of the clinical data viewer 414 is further illustrated in FIG. 11a. Battery tab 724 provides details about the battery life and charging patterns of the stimulator 100. The statistics are calculated using Usage Log events which log when charging starts and stops, and the battery voltage at each of these times. The following statistics are available:

| | |
|---|---|
| Number of | The total number of times the battery was charged |
| charges | in the specified period. |
| | Note: Only sessions that are more than 1 hour |
| | apart are counted as a seperate charge. |
| Total Charge | The total length of time spent charging. |
| Time | |
| Average Charge | The average duration of each charging session, i.e. |
| Duration | Total Charge Time/Number of Charges. |
| Time Between | The average duration of time between each |
| Charges | charging session. |
| Discharge Rate | The rate that the battery voltage drops, on |
| (average) | average (mV/hour). |
| Discharge Rate | The rate that the battery voltage drops, on |
| (stimulating) | average while stimulating (mV/hour). |
| Estimated Battery | The estimated battery life, for a single charge, |
| Life (average) | assuming that the patient's usage patterns remain |
| | the same. Note: the healthy battery range is |
| | between 4.1 V to 3.25 V. |
| Estimated Battery | The estimated battery life, for a single charge, if |
| Life (stimulating) | the stimulator is always stimulating. |
| Average Current | The average current draw while stimulating (mA). |
| Draw | The calculation is based on a battery capacity of |
| (stimulating) | 200 mAh. |
| Average Percent | The average percentage of full charge when the |
| Charged | patient stops charging. If the patient always |
| | charges until full charge this value would be 100%. |
| Average Percent | The average percentage of full charge when the |
| Discharged | patient starts charging. If the patient always lets |
| | the battery run out before charging this value |
| | would be 0%. |

To produce the battery discharge rate calculation for each discharge interval, the total time spent with stimulation on is calculated. The duration with stimulation enabled and the total duration of the interval is then plotted against the total voltage change for that interval, as shown in FIG. 11b. The red circles represent a single discharge interval, with the orange cross representing the total time and the blue cross representing the total time stimulating in that interval. Note that the total time is always greater than the time stimulating, and the difference represents the duration spent with stimulation off

Two lines-of-best-fit are then calculated through the points of the total duration and duration of stimulation, as shown. The line of best fit is calculated using the formula:

$$m = \frac{Σ x y - \frac{Σ x Σ y}{n}}{Σ {x}^{2} - \frac{{( \sum x )}^{2}}{n}} \\ b = \frac{Σ y}{n} - \frac{m Σ x}{n}$$

The discharge rates are then taken from the gradient of the corresponding line-of-best-fit. The battery life is calculated based on a healthy battery voltage between 4.1 V to 3.25 V, a range of 850 mV. The battery life is calculated by dividing the discharge rate by the healthy voltage range. A battery life estimate is calculated based on average usage as well as just stimulating. The average current draw is calculated by dividing the battery capacity (200 mAh) by the calculated battery life while stimulating.

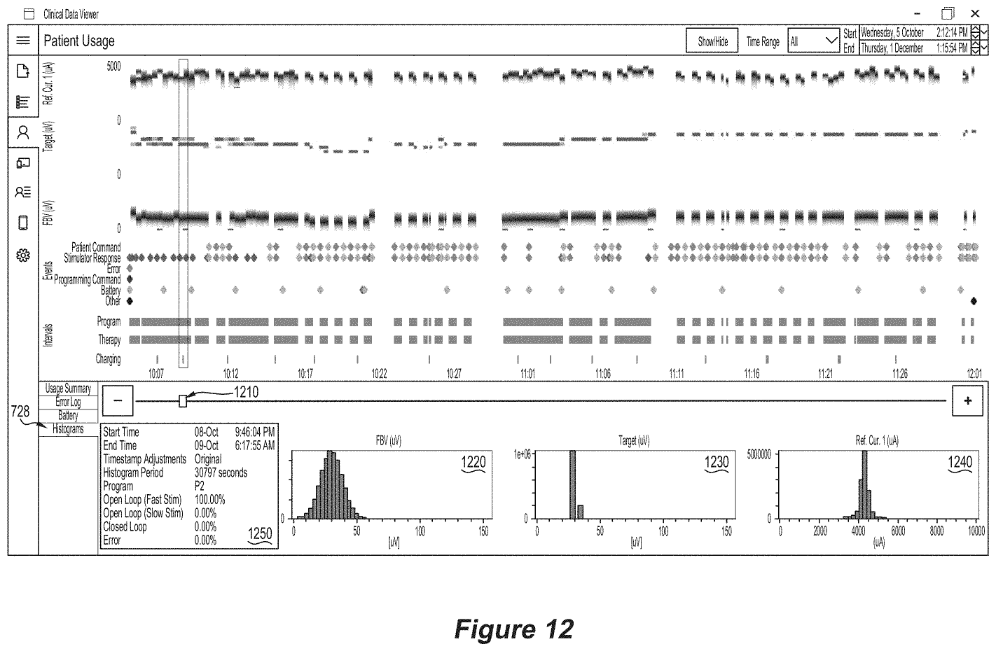

FIG. 12 illustrates the Histograms tab 728 of the clinical data viewer 414. Within Histograms tab 728, slider bar 1210 is a graphical user interface which allows the reviewer to select a subset of the 8 weeks of data. For that selected subset of data, the clinical data viewer 414 extracts histogram data for the feedback variable 1220, feedback target 1230, and stimulus current 1240 for closer assessment by the reviewer in tab 728. The histogram extracted from each short period of data is also presented as Therapy Logs 710, which show the individual histograms over the entire 8 week period, using a heat-map to efficiently represent the column heights of each histogram 1220, 1230, 1240.

The Histograms tab 728 allows each individual histogram in the Therapy Log to be displayed. Histograms are grouped by time period. Histogram recording begins immediately when stimulation is started, and stops when stimulation is stopped. The time period is selected by adjusting the slider 1210 or pressing the +or − buttons. When the time period is changed, the following items are automatically updated: (a) the Individual histogram plots 1220, 1230, 1240 whereby the number of individual histogram plots displayed depends on the number of stim set cycles that were in use at that time; (b) the Histogram time window shown by yellow highlight on the Therapy Log heat map, and (c) Statistics, being Histogram metadata that applies to all the histograms being displayed. Statistics are shown at 1250 and include Start Time, End Time, Timestamp Adjustments (Original or Adjusted, the latter to indicate that the timestamps were adjusted to correct instances where the timestamp was reset due to a power loss), Histogram Period, Program, Stimulator Mode % (Open Loop, Closed Loop, Error).

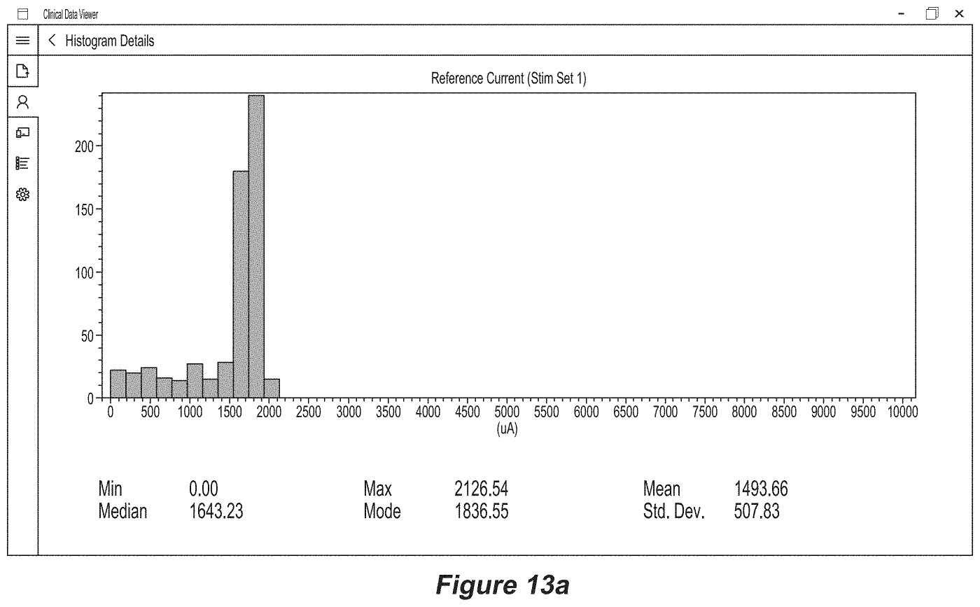

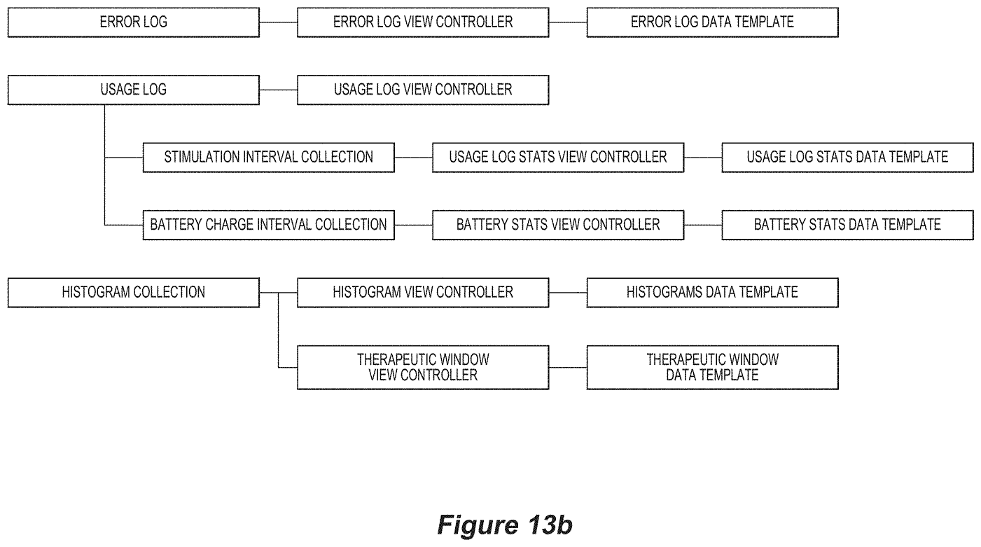

As shown in FIG. 13b the Histogram View Controller contains logic used to generate the histogram heat-maps 710 as well as display individual histogram plots 1220, 1230, 1240. Since the HistogramCollection may contain different types of histograms (i.e. FBV, CV, Target) it is the responsibility of the Histogram View Controller to filter the list to display the correct histograms.

To obtain more details or to zoom in on an individual histogram, the reviewer can double-click the histogram display. This will open the Histogram Details page, shown in FIG. 13a. In addition to displaying an enlarged version of the histogram, The Histogram Details page also displays statistics including the minimum value, maximum value, mean, median, mode, and standard deviation.

The different view controllers and their related models and views are shown in FIG. 13b. When a new file is loaded, the selected data logs are merged and then passed into the constructor of the relevant UsageLog, HistogramCollection or ErrorLog class. For the Usage Log, extra processing may be done to fix the timestamps. Once these models are created, they can be passed to the constructor of their view controllers.

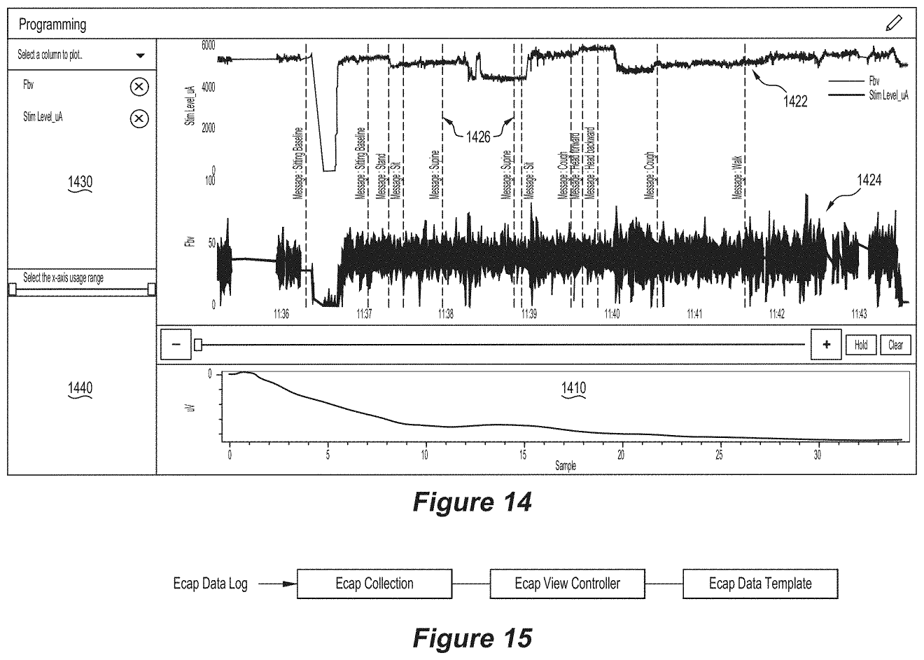

FIG. 14 illustrates the Programming Session Page. This displays ECAP trace(s) in the EcapDataTemplate 1410. The Programming Session Page is data bound to the Programming Session View Controller. The programming session page further displays Time-Series Data such as stimulation current level 1422 and the feedback variable (ECAP amplitude) 1424, with such traces being selectable in the DataLogColumns section 1430 and for example other traces such as Programs, Feedback Statuses, Impedances, Temperatures, Stimulator Info may additionally or alternatively be selected for display as times series data. Programming Notes 1426 are also displayed overlayed upon the time series plots. Program parameters which gave rise to the ECAP trace 1410 may be displayed in the ProgramDataTemplate window 1440.

The components involved in displaying ECAPs are shown in FIG. 15. ECAPs are displayed at 1410 using an EcapDataTemplate (defined in DataTemplates.xaml) which is bound to an EcapViewController which holds a reference to an EcapCollection. The EcapCollection holds a reference to an Ecap Data Log in the Clinical Data Log file. When requested by the EcapViewController, the EcapCollection reads a single ECAP from the Ecap Data Log and returns a new ECAP object. Using this “Lazy Loading” approach (i.e. only reading data into memory when required) the Clinical Data Viewer is able to minimise memory consumption.

Programming Notes 1426 are all read from the Clinical Data Log file when the Notes Data Log is passed to the constructor of the NoteLog class. The NoteLog contains a collection of Note objects which each contain a timestamp and a Dictionary object. Notes 1426 are displayed in the form of LineAnnotations on the main Stimulus Domain Plot.

The Programming page of FIG. 14 displays data collected by the Clinical Programming Application 410 during a programming session (while the CPA 410 is connected to a stimulator 100). FIG. 6.1 shows the default state of the page when a Clinical Data Log file is opened. The field at top left allows columns to be added or subtracted from the plot. The main frame at top right presents the chosen time-series plots. The frame at bottom left assesses posture change. The plot at the bottom shows the plot of a single ECAP recording chosen from the large number of recordings available.

Data collected during programming is written into five different tables

| | |
|---|---|
| Feedback | Each row of this table corresponds to a single stimulation. |
| Statuses | Columns include the stimulation current, feedback |
| | variable and feedback target, among others. |
| Programs | Each time a program is sent to the stimulator or a |
| | program parameter is changed (e.g. the current is |
| | increased) a new row is written to this table. |
| Impedances | Contains the results of each impedance check. |
| Temperatures | The CPA periodically records the stimulator temperature |
| | in this table. |
| Stimulator | This table contains data relating to the stimulator but not |
| info | pertaining to the therapy, such as the battery voltage, |
| | active program and system state. |

Programming notes 1426 entered using the CPA 410 are displayed on the time series plots of FIG. 14. A dashed vertical blue line denotes the exact time when that note was recorded. The notes 1426 can be hidden or displayed by toggling the notes button in the upper left corner of the screen. The Programming Tab also allows the user to calculate statistics on data collected during a programming session, which typically consists of a number of directed postural transitions where each posture is held for a short period or where current is varied over time for a set posture.

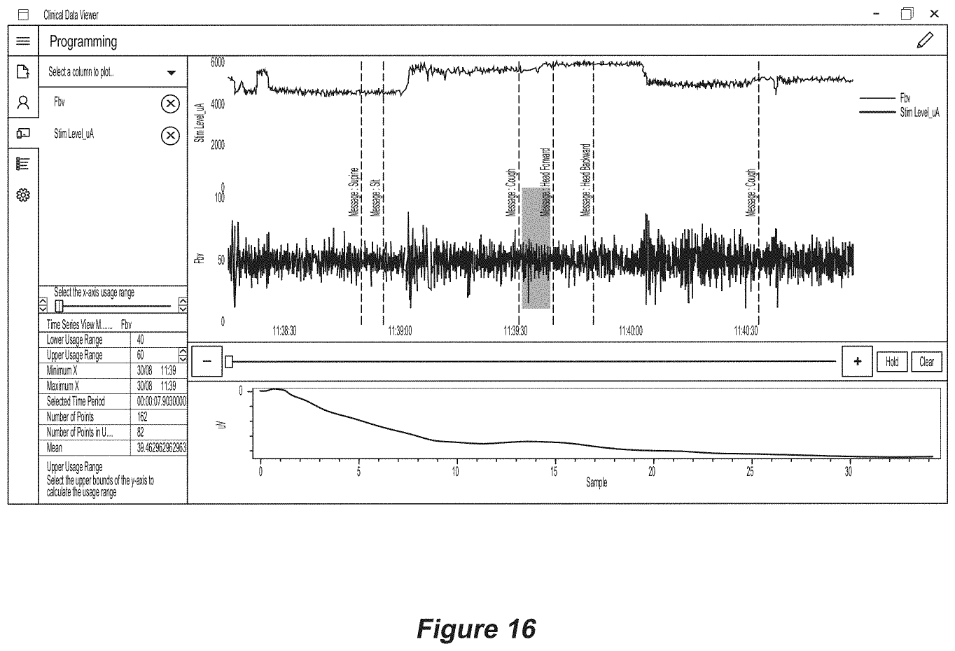

For example, FIG. 16 displays how to calculate the number of stimulations where the FBV falls within a certain range (which may be the patient's preferred usage range). The process proceeds as follows:
1. Select the data (under the “Select a column to plot . . . ”) that you would like to calculate the statistics for (in this example, Fbv). When a column is clicked, a shaded annotation (translucent green in this example) is added to the chart to highlight the data being selected.
2. Select the time range to calculate the statistics for by dragging the range slider or using the up/down buttons next to the slider. Notice that the width of the annotation (shaded green area on the plot) now shrinks to show the area selected.
3. Enter the upper (maximum) and lower (threshold) usage range. In this example, the patient has a threshold of 40 uV and a maximum of 60 uV.
4. Extract usage statistics from the table. Number of Points in Usage Range indicates how many of these stimulations were within the selected usage range. In this example, out of 162 stimulations in this time window, 82 were between 40 and 60 uV. The mean Fbv, as a result of those 162 stimulations, was 39.5 uV.

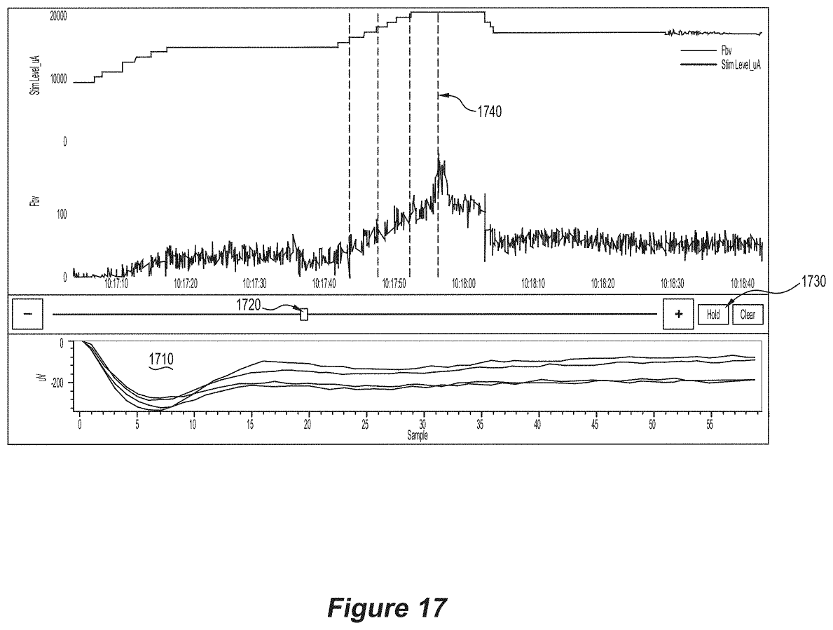

The ECAP plot of FIG. 17 is a component of the clinical data viewer 14 which allows the user to view individual ECAP traces 1710 recorded during the programming session. The particular ECAP or ECAPs being displayed can be changed by pressing the +or 31 buttons to step through the large number of ECAPs in the file, or by dragging the slider 1720. Multiple ECAPs 1710 can be compared by pressing the “Hold” button 1730 to lock the current ECAP to the plot. Held ECAPs are cleared by pressing the “Clear” button.

The time that an ECAP was produced is annotated by a vertical line on the time-series plot, one example being denoted 1740. Each held ECAP is annotated on the time-series plot. The colour of the annotation 1740 matches the colour of the ECAP trace 1710.

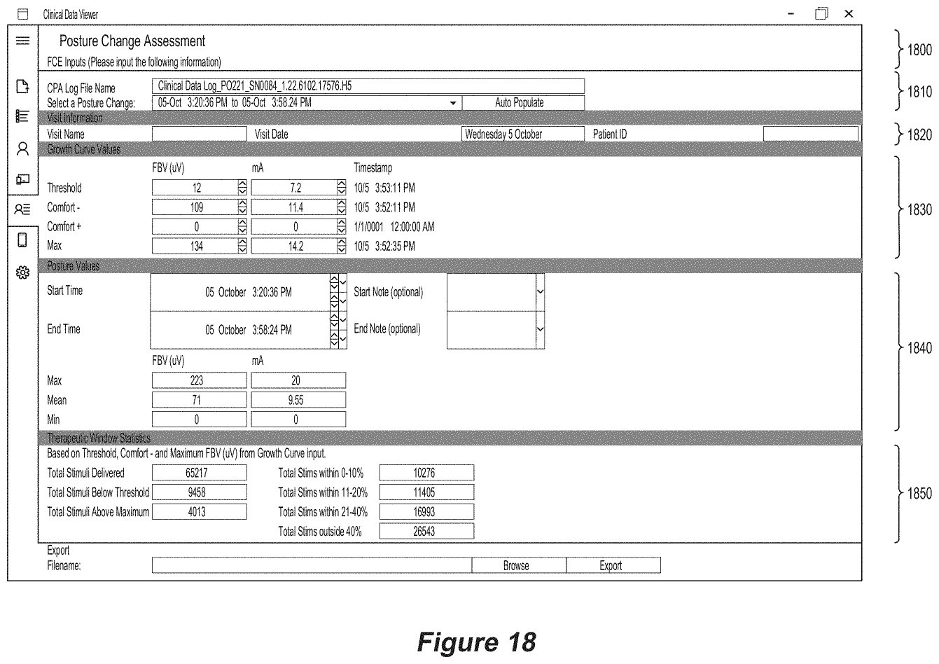

FIG. 18 illustrates the Posture Change Assessment page of the clinical data viewer 414, which displays data collected by the Clinical Programming Application 410 during a programming session (while the CPA is connected to a stimulator 100). It uses a combination of notes and logs of certain commands entered by the user in the CPA 410 during a programing session to auto-populate several of the fields. It then allows the user to calculate information about the FBV (uV) and current (mA) for user defined time and amplitude ranges. FIG. 18 shows the default state of the page when there is programming data in the Clinical Data Log file that was opened.

The Posture Change Assessment page assumes that during a programming session a Growth Curve is calculated for the patient in a single static posture, and each level (Threshold, Comfort−, Comfort+, and Maximum) is marked in the CPA 410 or recorded elsewhere, and that the patient is asked to undergo a series of posture changes and each is marked in the notes field (prior to each indicated posture change). The Posture Change Assessment page is split into 6 sections, including FCE (field clinical engineer) Inputs 1810, Visit Information 1820 and Growth Curve Values 1830 which describes the Growth Curve, which determines the Therapeutic Window of the patient (defined as the FBV between Threshold and Max, inclusive).

Growth Curve Values section 1830 has 8 fields, in pairs of 2, as follows. Threshold (uV and mA): The data in this field is auto generated based on the selected posture change time (“Start Time” and “End Time”) and when the ‘T’ button was last pressed during that time frame. Comfort− (uV and mA): The data in this field is auto generated based on the selected posture change time and when the ‘C−’ button was last pressed during that time frame. Comfort+(uV and mA): The data in this field is auto generated based on the selected posture change time and when the ‘C+’ button was last pressed during that time frame. Max (uV and mA): The data in this field is auto generated based on the selected posture change time and when the ‘M’ button was last pressed during that time frame. Each of these fields can also be edited by the user in the clinical data viewer 414. This allows for more freedom in the analysis. For example, it enables analysis of data where notes may have been written down, instead of entered into the CPA, during the programming session. Each field has an increment/decrement button next to it with a resolution of 1 uV or 1 mA depending on the field. Each field has a timestamp that denotes where in the log file the data was auto-populated from. If any data in the fields is modified by the user, the timestamps are removed and replaced with a message indicating that the values have been manually adjusted.

Posture Values section 1840 allows the user to select a window (during the programming session) within which to calculate some statistics (min, max and mean) on the FBV (uV) and current (mA). This section has 8 fields, as follows. Start Time: The start of the analysis window. Auto-populated from the posture change Start Time above. This can also be edited by using the arrows to the right of the timestamp or by selecting a “Start Note” from the dropdown further to the right. Note: This can be any note logged to the file, it does not need to be related to a posture change. End Time: The end of the analysis window. This works in the same way as the Start Time described above. Max (uV and mA): The maximum FBV (uV) and current (mA) within the selected window. Mean (uV and mA): The average FBV (uV) and current (mA) within the selected window. Min (uV and mA): The minimum FBV (uV) and current (mA) within the selected window.

The Therapeutic Window Statistics section 1850 contains several calculations for the FBV within the time window, during a programming session, defined under Posture Values. Calculations are based on the values of Threshold, Comfort− (not Comfort+) and Maximum under Growth Curve Values. This section has 7 fields (the values are auto-populated and cannot be edited by the user), as follows. Total Stimuli Delivered: The total number of stimuli delivered in the time window. Total Stimuli Below Threshold: The number of stimuli delivered where the FBV was below Threshold (uV). Total Stimuli Above Maximum: The number of stimuli delivered where the FBV was above Maximum (uV). Total Stims within 0-10%: The number of stimuli delivered where the FBV was within ±10% of the Comfort− (uV) value (≥ to 90% and ≤110% of Comfort−). Total Stims within 11-20%: The number of stimuli delivered where the FBV was >110% and ≤120% of Comfort− or ≥80% and <90% of Comfort−. Total Stims within 21-40%: The number of stimuli delivered where the FBV was >120% and ≤140% of Comfort− or ≥60% and <80% of Comfort−. Total Stims outside 40%: The number of stimuli delivered where the FBV was >140% or <60% of the Comfort− (uV) value.

The Export section of the Posture Change Assessment page allows the user to export a .csv file or the like.

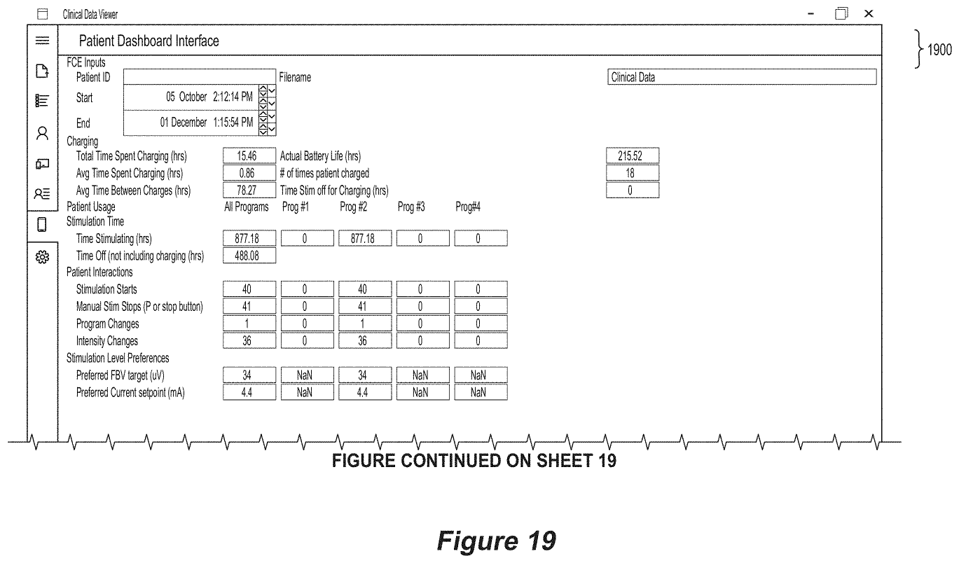

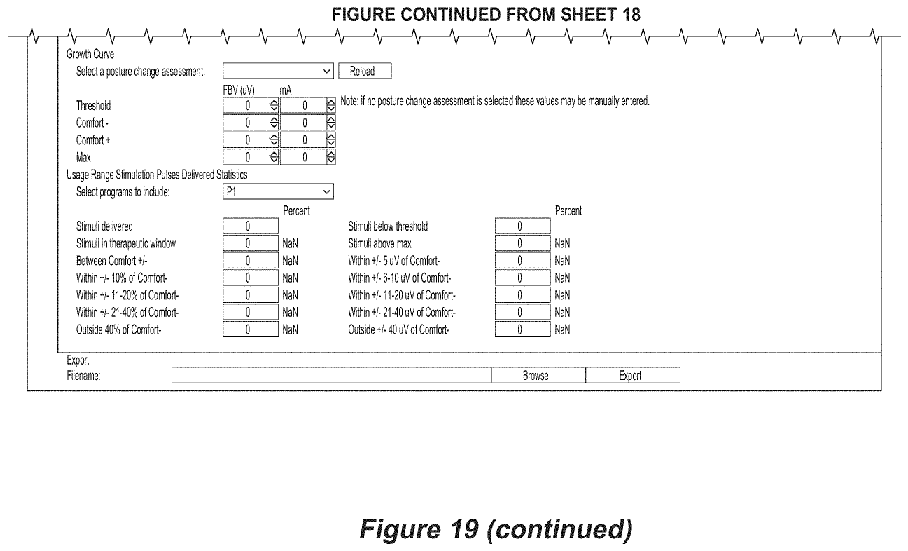

FIG. 19 illustrates the Patient Dashboard Interface page of the clinical data viewer 414, which is a comprehensive summary of device usage statistics, within a given time period, using growth curve data, and using the Therapy Logs downloaded from the stimulator 100. The page contains several fields that can be auto-populated from the log data; some can also be edited by the user (orange shaded boxes). Boxes with a white background are auto-populated and cannot be edited by the user.

The CDV 414 further provides for correction of timestamps that are incorrect due to the stimulator battery running out. Note that if the battery ran flat more than once between programming sessions, only the most recent events will be corrected. Alternatively, the CDV shifts timestamps so that the timestamps of items in the Usage and Error Log or Histogram are ordered chronologically. This will introduce an error into the timestamps but will mean that the events can be displayed in the correct chronological order. The total error will be equivalent to the amount of time that the stimulator battery was flat for before charging commenced.

Zooming and panning of each chart of the CDV 414 is provided, as appropriate to the input device:

| Input | Zoom | Pan |
|---|---|---|
| Touch | Two-finger “pinch and zoom”. | Single-finger |
| screen | | drag. |
| Track pad | To finger “pinch and zoom” or use | Right-click and |
| | two fingers and drag upwards to zoom | drag. |
| | in or downwards to zoom out | |
| Mouse with | Zoom in and out by rotating the scroll- | Right-click and |
| scroll-wheel | wheel. | drag. |
| | Alternatively, select the area to zoom to | |
| | by holding the scroll-wheel button or | |
| | right-mouse button) and dragging the | |
| | mouse. | |

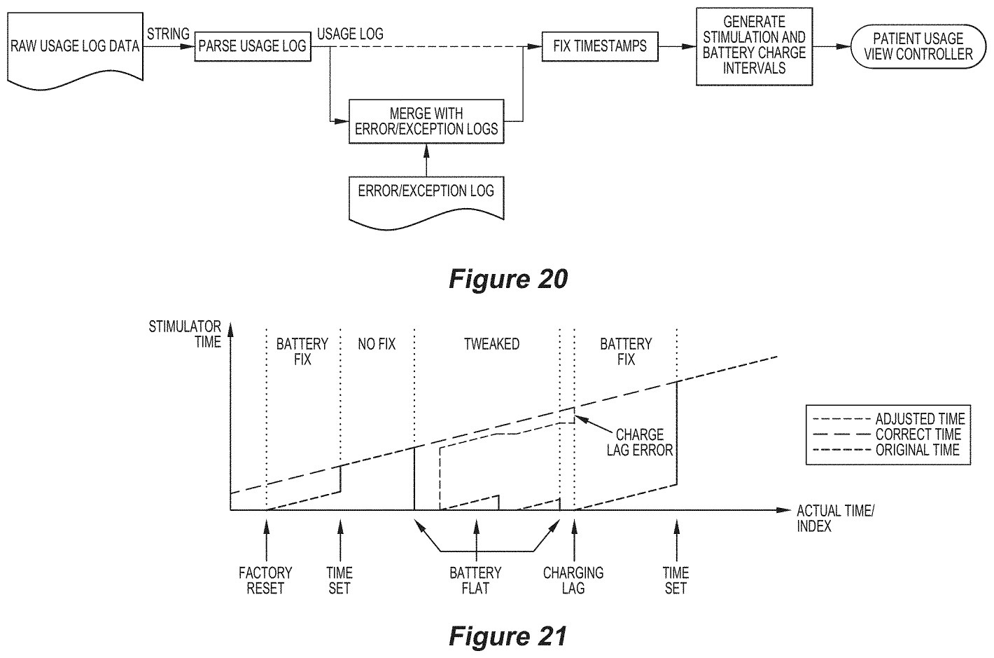

The data in the raw text file of the usage log must be processed in several stages before being displayed by the Clinical Data Viewer 414. The high-level process is outlined in FIG. 20. The Parse Usage Log converts the raw Usage Log text string into a UsageLog object. If an Error/Exception Log is available it can be merged with a Usage Log. This allows more detailed statistics to be calculated (e.g. when calculating the reason for the stimulation stopping) as the Usage Logs alone do not contain error events specific to the therapy. The timestamps that are stored in the Usage Logs may be incorrect due to the battery running flat. There are two methods of timestamp correction. A first function works by iterating through the Usage Log looking for events that indicate that the time was set by the CPA 410. When the time set event is found, the error can be determined from the difference between the values of the “Timestamp” and “PreviousTimestamp” fields of that event. Not all timestamps can be fixed by the “Battery Fix”. If the battery runs flat more than once, only the interval up to the last flat battery can be fixed. The remaining incorrect timestamps are adjusted so that they are in the chronological, however this will mean that there will still be some error which is proportionate to the amount of time spent with a flat battery. The error is named the “Charge Lag Error” in FIG. 21.

The process of FIG. 20 next proceeds to generate Stimulation and Battery Charge Intervals. The data can be interpolated so that the state of the device can be determined for the interval between each event. The constructor of the StimulationlntervalCollection class iterates through items in the Usage Log and adds Stimulationlntervals based on a series of state variables.

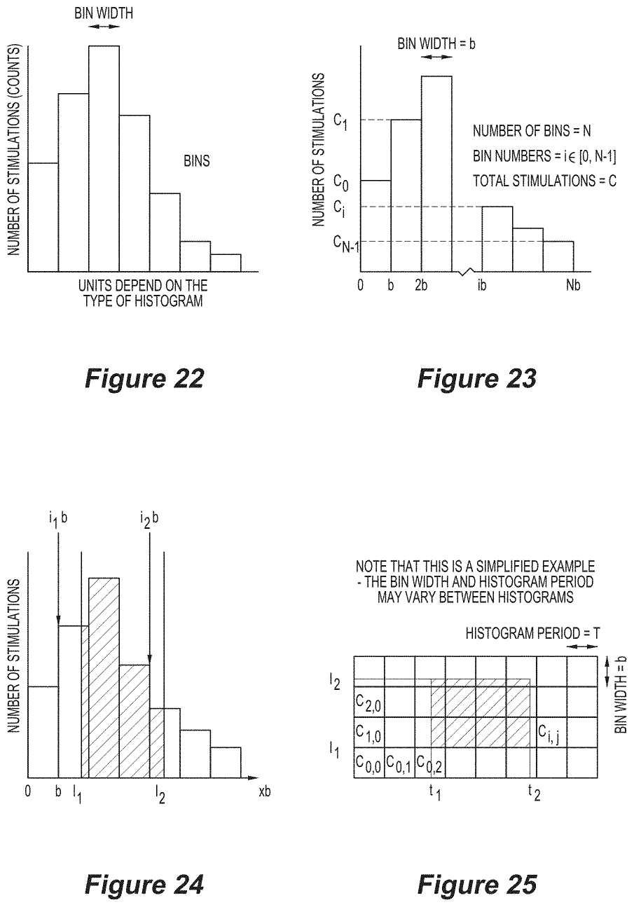

As noted previously the Clinical Data Viewer 414 also parses histograms obtained from the Firmware and allows them to be plotted individually or together in the form of a heat-map. This is addressed further in FIGS. 22-25 and the following. The table below describes how statistics are calculated on individual histograms. Note that the units (except for the Total Number of Stimulations) are the same units as used by the x-axis, i.e. uV for the FBV and FBV Target and uA for the CV.

| | |
|---|---|
| Histogram | A list of Histograms, with additional methods to calculate statistics |
| Collection | over a specified time period and multiple histograms. |
| Histogram | Histograms group the stimulations produced in a given time window |
| | by a specified parameter. |
| Start Timestamp | The time that recording started. |
| End Timestamp | The time that recording finished. |
| Therapy States | An array of counts that represent the number of stims that were |
| | produced for each system state. The sum of the elements equals |
| | the total number of stimulations produced between the Start |
| | Timestamp and End Timestamp. |
| | The index of the array represents the system state: |
| | 0: Open Loop (Fast Stim) |
| | 1: Open Loop (Slow Stim) |
| | 2: Closed Loop |
| | 3: Closed Loop (Error) |
| Group Id | Histograms that are being recorded simultaneously have the same |
| | Group ID. The Group ID is incremented when the histogram fills up |
| | or stimulation stops. |
| No. of Histograms | Indicates the number of histograms in the group. For example, if the |
| in the Group | running program has 2 stim sets, there will be 4 histograms in the |
| | group: Feedback Variable, Feedback Target, Reference Current |
| | Cycle 1 and Reference Current Cycle 2. |
| Id in the | Indicates the data that is stored in the histogram. |
| Group/Type | 0 = Feedback Variable (bin units: μV) |
| | 1 = Feedback Variable Target (bin units: μV) |
| | 2 = Reference Current Cycle 1 (bin units: μA) |
| | 3 = Reference Current Cycle 2 (bin units: μA) |
| | 4 = Reference Current Cycle 3 (bin units: μA) |
| | 5 = Reference Current Cycle 4 (bin units: μA) |
| | 6 = Total Current Cycle 1 (bin units: μA) |
| | 7 = Total Current Cycle 1 (bin units: μA) |
| | 8 = Total Current Cycle 1 (bin units: μA) |
| | 9 = Total Current Cycle 1 (bin units: μA) |
| Program Id | Indicates the program index (0-3) that was used to generate the |
| | histogram. Each histogram in the group will have the same Program |
| | Id. |
| Bin Width | Specifies the range of the unit for each bin (b), see Error! Reference |
| | source not found.. The bin width is calculated from a numerator (n) |
| | and a denominator in bit shifts (d) field using the formula: |
| | $b = \frac{{2}^{d}}{n}$ |
| Number of Bins | The number of elements in the “Bins” array. |
| Bins | An array of stimulations for a given range. The range is calculated |
| | using the formula: |
| | [ib, (i + 1)b) |
| | where i is the index of the element in the array and b is the bin |
| | width. |
| Errors | Flags which indicate whether the following errors occurred while |
| | the histogram was being recorded: |
| | 0×01-Reference Clock Error occurred |
| | 0×02-Out of Compliance occurred |
| | 0×04-Possible Noise occurred |
| | 0×08-ERT Signal Clipping occurred |
| CRC | CRC of the histogram group. Only the last histogram in the group |
| | has a value that is not 0. The CRC algorithm used is the 16-bit CRC- |
| | CCITT algorithm implemented in the MSP430 F5659 hardware. |

Clinical Data Viewer Histogram definitions and
parameter information

| | |
|---|---|
| Total Number of Stimulations | $C = {\sum }_{i = 0}^{N - 1} {c}_{i}$ |
| Minimum (left-hand-edge of | ib for min i where cᵢ > 0 |
| the smallest bin with a count | |
| greater than 0) | |
| Maximum (right-hand-edge | (i + 1)b for max i where cᵢ > 0 |
| of the largest bin with a | |
| count greater than 0) | |
| Mean (μ) | $μ = \frac{1}{C} {\sum }_{i = 0}^{N - 1} {c}_{i} ( \frac{ib + ( i + 1 ) b}{2} )$ |
| Median (M_{d}) | ${\sum }_{i = 0}^{{M}_{d} / b} {c}_{i} = \frac{C + 1}{2}$ |
| Mode (M₀) | ${M}_{0} = ( \frac{ib + ( i + 1 ) b}{2} )$ |
| | $for i where {c}_{i} = {max}_{i ∈ [ 0 , N - 1 ]} {c}_{i}$ |
| Standard Deviation (σ) | $σ = \sqrt{\frac{1}{N} {\sum }_{i = 0}^{N - 1} {{c}_{i} ( ( \frac{ib + ( i + 1 ) b}{2} ) - μ )}^{2}}$ |

The clinical data viewer 414 is further configured to provide automated outputs to queries such as “what percentage of the time was the feedback variable within X % of the comfort level?” This problem can be generalised to calculating the number of counts within a given window, such as the window [l₁,l₂] in FIG. 24. Note, the calculation of the number of counts in this window assumes that the distribution of values within a bin is uniform, which will typically not be precisely true, however making this assumption greatly improves the efficiency of the calculation.

The first step in solving this problem is determining the “bin index”, x, for the edge values:

l₁=x₁b, x₁, bϵ [symbol] , xϵ[0, N]
l₂=x₂b, x₂, bϵ [symbol] , xϵ[0, N]
where x is a real number restricted to the range [0, N]. If l is such that x<0 or x>N then x is clipped. In the example above, x₁=1.75 and x₂=4.5.

Next, the integer bin index, i, that l₁ and l₂ fall in are calculated:

i₁=floor(x₁), i₁ϵ [symbol]
i₂=floor(x₂), i₂ϵ

[symbol]

In the example above, i₁=1 and i₂=4. The fraction of the bins being used can then be calculated:

$${r}_{1} = \frac{( {i}_{1} + 1 ) b - {l}_{1}}{b} \\ {r}_{2} = \frac{{l}_{2} - {i}_{2} b}{b}$$

In the example above, r₁=0.25 and r₂=0.5. Finally, the sum of the bins can be calculated:

$${C}_{1 , 2} = part of first bin + sum of center bins + part of last bin = {r}_{1} {c}_{{i}_{1}} + {\sum }_{i = {i}_{1} + 1}^{{i}_{2} - 1} {c}_{i} + {r}_{2} {c}_{{i}_{2}}$$

An extension to the question posed above is “what percentage of the time was the feedback variable within X % of the comfort level for the last 3 months?” To answer questions like this, the number of counts in a range must be aggregated across multiple histograms.

The algorithm for calculating this statistic is similar to that described above. Say that the number of stims between 11 and 12 and was to be calculated for the time period t1 to t2. The algorithm is as follows:
1. For each histogram set in the list of histograms, calculate how much of that histogram's time span is within the period [t₁, t₂].
2. Calculate the number of stimulations between l₁ and l₂ using the algorithm described above.
3. Multiply the proportion calculated in step (0) by the count in step (0).
4. Also calculate the total number of stimulations for each histogram and multiply by the proportion from step (0).
5. Add the counts from step (0) to determine the number of stims that occurred within the range and from step (0) to determine the total number of stims for that time period.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

1. A system for managing clinical data of an implanted neuromodulation device, the system comprising:
an implanted neuromodulation device configured to deliver electrical neurostimulation therapy, and configured to capture recordings of neural responses to the electrical neurostimulation therapy, and configured to communicate clinical data including recordings of neural responses to a non-implanted receiver; and
a supervisory device configured to receive clinical data from the implanted neuromodulation device via the receiver, and to present the clinical data for review by a reviewer.

2. The system of claim 1 wherein the implanted neuromodulation device is configured to record the clinical data substantially continuously during device operation.

3. The system of claim 1 wherein the implanted neuromodulation device is configured to communicate all recorded clinical data to the non-implanted receiver whenever within wireless communications range of the non-implanted receiver.

4. The system of claim 1 wherein the implanted neuromodulation device is further configured to record clinical data comprising user commands input to the implanted neuromodulation device.

5. The system of claim 1 wherein the implanted neuromodulation device is further configured to record clinical data comprising at least one of stimulus current, feedback target and battery state.

6. The system of claim 1 wherein the supervisory device is configured to present the clinical data for review by presenting a subset of the clinical data as selected by filtering by time range.

7. The system of claim 1 wherein the supervisory device is configured to present statistics derived from the clinical data.

8. The system of claim 1 wherein the clinical data comprises logged events, wherein the supervisory device is configured to present events for review by an indicia, and wherein each such indicia may be clicked or tapped to reveal a summary or full detail of the respective logged event represented by that indicia.

9. The system of claim 1 wherein the supervisory device is configured to display therapy logs.

10. The system of claim 9 wherein a therapy log comprises a plurality of histograms of a therapy parameter.

11. The system of claim 10 wherein the histograms are represented on a timescale by use of a heat map representation of the histograms.

12. The system of claim 10 wherein the therapy parameter is an ECAP magnitude.

13. The system of claim 10 wherein the therapy parameter comprises a feedback target.

14. The system of claim 10 wherein the therapy parameter comprises a stimulus current value.

15. The system of claim 1 wherein the presented clinical data includes device programming data.

16. The system of claim 15 wherein the presented clinical data includes programming notes entered by a clinician during device programming.

17. The system of claim 1 wherein the presented clinical data includes quantitative analysis of one or more histograms of a therapy parameter.

18. A method of managing clinical data of an implanted neuromodulation device, the method comprising:
an implanted neuromodulation device delivering electrical neurostimulation therapy;
the implanted neuromodulation device capturing recordings of neural responses to the electrical neurostimulation therapy;
communicating clinical data including the recordings of neural responses to a non-implanted receiver; and
receiving the clinical data from the implanted neuromodulation device via the receiver, and presenting the clinical data for review by a reviewer.

19. (canceled)

20. A non-transitory computer readable medium for managing clinical data of an implanted neuromodulation device, comprising instructions which, when executed by one or more processors, causes performance of the following:
receiving clinical data from the implanted neuromodulation device, the clinical data including recordings captured by the implanted neuromodulation device of neural responses to electrical neurostimulation therapy delivered by the implanted neuromodulation device; and
presenting the clinical data for review by a reviewer.

21. (canceled)
